(19) United States
(12) Patent Application Publication
(10) Pub. No.: US 2023/0364466 A1
(43) Pub. Date: Nov. 16, 2023

(54) ELECTRONIC DEVICE, EXERCISE DATA ACQUISITION METHOD, AND RECORDING MEDIUM

(71) Applicant: CASIO COMPUTER CO., LTD., Shibuya-ku, Tokyo (JP)

(72) Inventors: Tomoharu YAMAGUCHI, Kodaira-shi, Tokyo (JP); Hiroyasu IDE, Hino-shi, Tokyo (JP)

(73) Assignee: CASIO COMPUTER CO., LTD., Shibuya-ku, Tokyo (JP)

(21) Appl. No.: 18/022,997

(22) PCT Filed: Aug. 3, 2021
(86) PCT No.: PCT/JP2021/028781

§ 371 Date: Feb. 24, 2023

(30) Foreign Application Priority Data
Sep. 14, 2020 (JP) 2020-153469

(51) Int. Cl. A63B 24/00

(52) U.S. Cl. CPC A63B 24/0006; A63B 2220/10; A63B 2220/40; A63B 2220/836

(57) ABSTRACT

An electronic device includes a controller that acquires, from an acceleration sensor, acceleration data corresponding to a motion state in a body-lateral direction orthogonal to a body axis of a user that is moving by legs of the user, and; derives velocity data based on the acceleration data and, also, derives an error of the velocity data based on an average value of the velocity data of a period that is a multiple of a first two-step cycle and an average value of the velocity data of a period that is a multiple of a second two-step cycle that directly precedes or follows the period that is a multiple of the first two-step cycle, and corrects the velocity data using the error of the velocity data to generate corrected velocity data.

### TECHNICAL FIELD

The present disclosure relates to an electronic device, an exercise data acquisition method, and a program.

### BACKGROUND ART

When performing exercise such as running or the like, an inertia sensor is attached to the waist of a user to acquire exercise data, and analyses of the movements of the body, such as running form and the like, during exercise are carried out.

Patent Literature 1 describes an exercise analysis device that calculates the left-right movement of a user on the basis of measurement data acquired from an inertia sensor attached to a user.

### CITATION LIST

### Patent Literature

Patent Literature 1: Unexamined Japanese Patent Application Publication No. 2016-32611

### SUMMARY OF INVENTION

### Technical Problem

However, when attempting to obtain desired data on the basis of certain data, error accumulates as a result of measurement error, direction estimation accuracy, drift due to integration, and the like, and it is difficult to precisely and stably calculate the left-right movement.

The present disclosure is made with the view of the above situation, and an objective of the present disclosure is to precisely and stably calculate the left-right movement of a user during exercise.

### Solution to Problem

An electronic device according to the present disclosure includes:
a controller, wherein
the controller is configured to
   acquire, from an acceleration sensor, acceleration data corresponding to a motion state in a body-lateral direction orthogonal to a body axis of a user that is moving by legs of the user, and
   derive velocity data on the basis of the acceleration data and, also, derive an error of the velocity data based on an average value of the velocity data of a period that is a multiple of a first two-step cycle and an average value of the velocity data of a period that is a multiple of a second two-step cycle that directly precedes or follows the period that is a multiple of the first two-step cycle, and correct the velocity data using the error of the velocity data to generate corrected velocity data.

### Advantageous Effects of Invention

According to the present disclosure, it is possible to precisely and stably calculate the left-right movement of a user during exercise.

### BRIEF DESCRIPTION OF DRAWINGS

FIG. 1 is a front view illustrating the appearance of an electronic device according to an embodiment of the present disclosure;

FIG. 2 is a drawing illustrating a state in which the electronic device according to the embodiment of the present disclosure is affixed to a human body;

FIG. 3 is a drawing illustrating the configuration of the electronic device according to the embodiment of the present disclosure;

FIG. 4 is a drawing explaining three axial directions of a gyrosensor and an acceleration sensor according to the embodiment of the present disclosure;

FIG. 5 is a drawing illustrating the functional configuration of a controller of the electronic device according to the embodiment of the present disclosure;

FIG. 6 is a flowchart illustrating the flow of left-right movement acquisition processing executed by the controller of the electronic device according to the embodiment of the present disclosure;

FIG. 7 is a flowchart illustrating the flow of attitude estimation processing executed by the controller of the electronic device according to the embodiment of the present disclosure;

FIG. 8 is a drawing illustrating a shift of the attitude in a direction of travel of the electronic device according to the embodiment of the present disclosure;

FIG. 9A is an angular velocity wave diagram, illustrating the shift of the attitude in the direction of travel of the electronic device according to the embodiment of the present disclosure;

FIG. 9B is an angle wave diagram illustrating the shift of the attitude in the direction of travel of the electronic device according to the embodiment of the present disclosure;

FIG. 10 is a flowchart illustrating the flow of left-right movement estimation processing executed by the controller of the electronic device according to the embodiment of the present disclosure;

FIG. 11 is a position wave diagram illustrating integration error of the electronic device according to the embodiment of the present disclosure;

FIG. 12 is a flowchart illustrating the flow of velocity data correction processing executed by the controller of the electronic device according to the embodiment of the present disclosure;

FIG. 13 is a flowchart illustrating the flow of position data correction processing executed by the controller of the electronic device according to the embodiment of the present disclosure; and

FIG. 14 is a time chart explaining the velocity data and the position data correction processing executed by the controller of the electronic device according to the embodiment of the present disclosure.

### DESCRIPTION OF EMBODIMENTS

Hereinafter, an electronic device according to embodiments of the present disclosure is described while referencing the drawings.

Note that, in the following, the term “run” refers generally to actions of moving using the legs of the user, and includes walking.

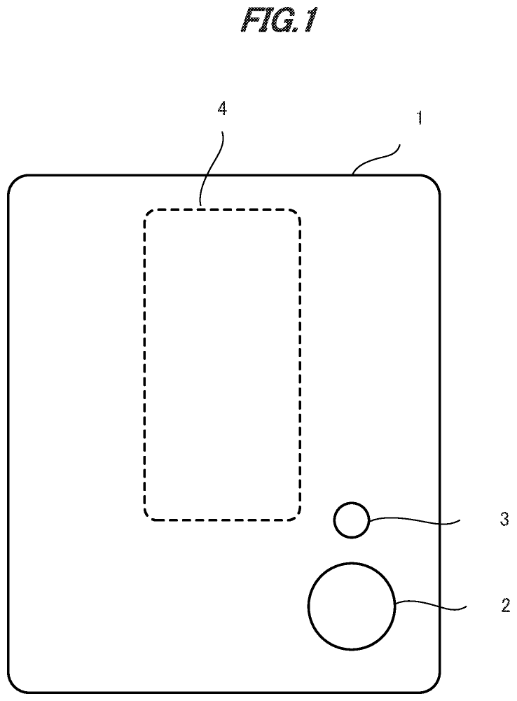

Various types of inertia sensors, described later, are integrated and built into an electronic device 1 according to the present embodiment. As illustrated in FIG. 1, the electronic device 1 includes a power key 2 on a front surface, and a display 3 configured from a light emitting diode (LED, for example. When the power key 2 is operated, acquisition of sensor data from the inertia sensors is carried out. The display 3 displays an operating state. For example, a lighted state of the LED is maintained while the power is ON and the acquisition of the sensor data from the inertia sensors is being carried out. Additionally, a clip 4 that is an attacher is provided on a back surface of the electronic device 1, and the electronic device 1 can be attached by clipping an object in the clip 4.

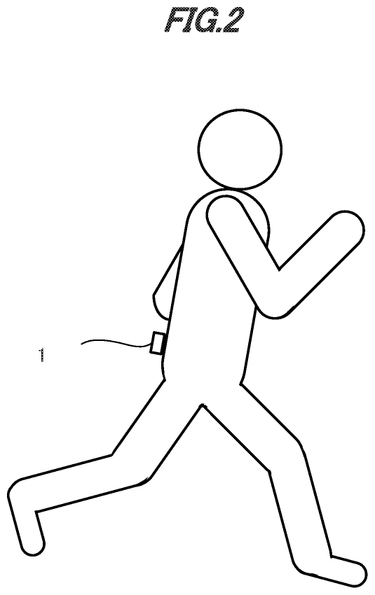

As illustrated in FIG. 2, for example, the electronic device 1 is affixed near the center of the waist on the back of the user at a time of running. The electronic device 1 is attached to the waist by clipping the clip 4 on an article of clothing or a belt of the user. As a result of being affixed to the waist of the user, the electronic device 1 acquires, by the built-in inertia sensors, movements of the body during exercise. Note that, provided that the electronic device 1 can precisely detect the movement of the torso including the core during exercise of the user, the electronic device 1 is not limited to being affixed to the waist and may be affixed tightly to a desired location of the human body such as the center of the abdomen or the chest, the neck, or the like. Additionally, provided that the electronic device 1 can be tightly affixed to the user, the attachment method is not limited to the clip 4, and a pin, adhesive tape, or the like may be used.

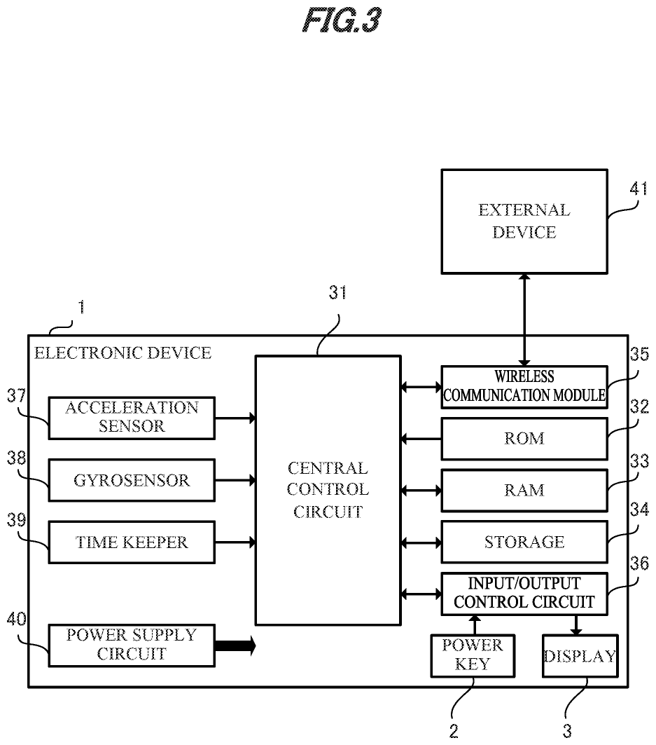

As illustrated in FIG. 3, the electronic device 1 includes a central control circuit 31 that controls the entire device, a read only memory (ROM) 32 that is a nonvolatile storage circuit, a random access memory (RAM) 33 that is a volatile storage circuit, a storage 34, a wireless communication module 35 that carries out wireless communication, an input/output control circuit 36 that controls input by the power key 2 and output to the display 3, an acceleration sensor 37 that detects acceleration, a gyrosensor 38 that detects angular velocity, a time keeper 39 that measures time, and a power supply circuit 40 that includes a battery such as a secondary battery or the like and that supplies power to the various circuits.

The acceleration sensor 37 and the gyrosensor 38 that are inertia sensors measure an exercise state of the user. The acceleration sensor 37 is a three-axis acceleration sensor, and measures changes of a motion velocity during exercise of the user by detecting acceleration in three axial directions that are orthogonal to each other.

The gyrosensor 38 is a three-axis angular velocity sensor, and measures changes of a motion direction during exercise of the user by detecting angular velocity rotating around each axis of the three axes that define the acceleration in the acceleration sensor 37.

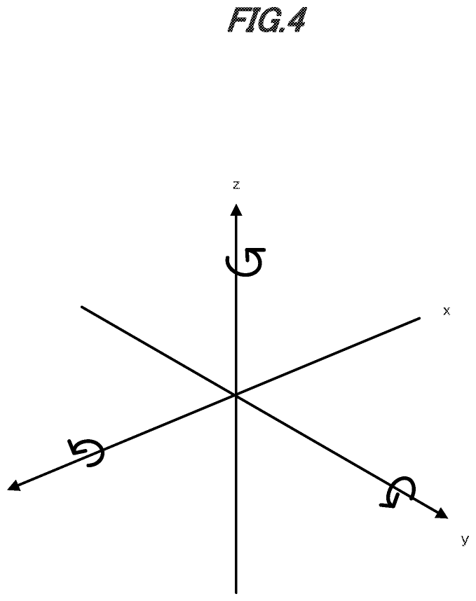

FIG. 4 is a drawing illustrating the three axial directions of the acceleration sensor 37 and the gyrosensor 38. A front-back direction of the human body during exercise such as running or the like is defined as a y-axis direction. Here, a direction of travel in which the user moves is defined as the + direction, and a direction opposite thereto is defined as the − direction. A left-right body-lateral direction of the human body that is orthogonal to the y axis is defined as an x-axis direction. Here, the right direction of the user is defined as the + direction, and a direction opposite thereto is defined as the − direction. A body axis direction that is a vertical direction of the human body and that is orthogonal to the x-y plane is defined as a z-axis direction. Here, an overhead direction of the user is defined as the + direction, and a direction opposite thereto is defined as the − direction. Angular velocity occurring in a right-wise direction toward the + direction of each axis is defined as the + direction.

The time keeper 39 measures elapsed time when acquiring the sensor data from the acceleration sensor 37 and the gyrosensor 38, and outputs the measured elapsed time as time data. Here, the time keeper 39 includes, for example, functions of a radio clock, and measures the elapsed time during exercise of the user with high accuracy on the basis of a standard radio wave transmitted from a transmitting station, or time information transmitted from a global positioning system (GPS) satellite. Additionally, the time keeper 39 may measure time using a basic clock generated by a built-in crystal oscillator.

The central control circuit 31 includes at least one processor, is connected to the various circuits via a bus, and executes control programs stored in the ROM 32 to realize various functions and control the entire device.

The ROM 32 stores the control programs for the central control circuit 31 to realize the various functions, and various types of fixed data. The RAM 33 functions as the working area of the central control circuit 31. The storage 34 is nonvolatile memory such as flash memory, a hard disk, or the like. The storage 34 stores programs used by the central control circuit 31 to carry out various processes, and data generated or acquired as a result of carrying out the various processes. The central control circuit 31 executes predetermined control programs to carry out control of detection operations of the acceleration sensor 37 and the gyrosensor 38, measurement operations of elapsed time of the time keeper 39, storage and reading operations to and from the RAM 33 and the storage 34 of the sensor data, transmission operations of exercise data to an external device 41 via the wireless communication module 35, and the like. Additionally, the central control circuit 31 performs attitude estimation processing and left-right movement estimation processing for the sensor data, and corrects the exercise data so that analysis/processing of the exercise data can be carried out correctly.

The wireless communication module 35 includes an interface that carries out communication with the external device 41 via a wireless local area network (LAN), Bluetooth (registered trademark), or the like, and carries out wireless communication with the external device 41 via a non-illustrated antenna. The exercise data acquired by the electronic device 1 is transmitted to the external device 41 via the wireless communication module 35. Note that the communication with the external device 41 may be carried out via a wired communication module such as a universal serial bus (USB) or the like instead of via the wireless communication module 35.

The input/output control circuit 36 converts a signal input from the power key 2 to data, and transmits the data to the central control circuit 31 and, also, controls the lighting of the display 3 on the basis of a control signal from the central control circuit 31.

The power supply circuit 40 includes a power supply integrated circuit (IC) and the like, and produces and supplies necessary power from the battery to the various circuits. Additionally, the power supply circuit 40 charges the battery.

The external device 41 receives the exercise data of the user transmitted from the electronic device 1 via the wireless communication module 35. The external device 41 analyzes the received exercise data and displays the analysis results. In one example, the external device 41 is a smartwatch worn by the user, a smartphone, a tablet terminal, a personal computer, a server device on a network, or the like. Specifically, the external device 41 may be a device that is carried or worn on the body such that the user can confirm the analysis results while exercising, or may be a device that the user does not carry and that is provided separately from the electronic device 1 such that the user can thoroughly confirm the analysis results after exercising.

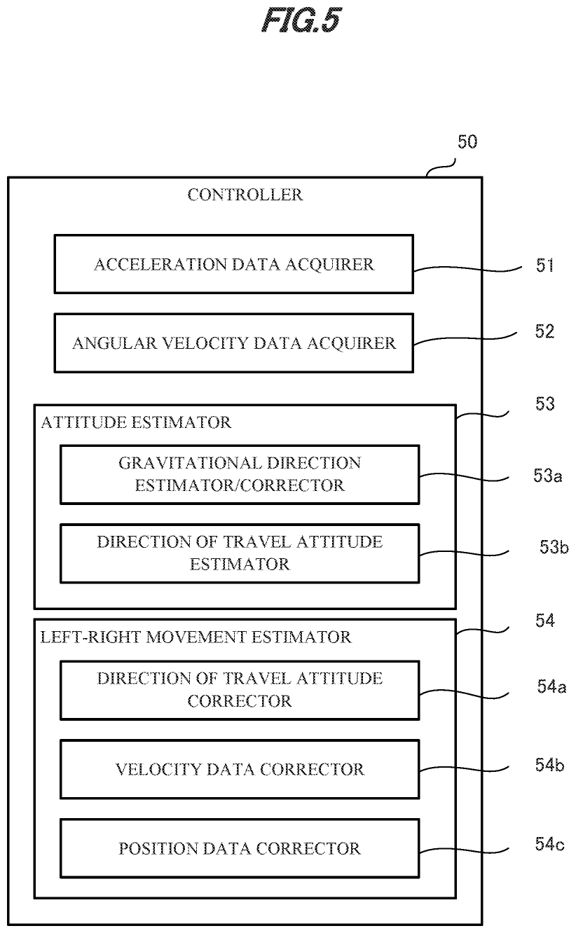

With the electronic device 1, the central control circuit 31 controls the operations of the various components in accordance with commands written in the program and, as illustrated in FIG. 5, the controller 50 that realizes the functions described hereinafter, is configured by software and hardware operating in cooperation.

The controller 50 includes an acceleration data acquirer 51 that acquires acceleration data from the acceleration sensor, an angular velocity data acquirer 52 that acquires angular velocity data from the gyrosensor, an attitude estimator 53, and a left-right movement estimator 54.

The acceleration data acquirer 51 acquires the acceleration data by sampling, on a predetermined sampling cycle, an acceleration signal detected by the acceleration sensor 37.

The angular velocity data acquirer 52 acquires the angular velocity data by sampling, on a predetermined sampling cycle, an angular velocity signal detected by the gyrosensor 38.

The attitude estimator 53 estimates, on the basis of the data of the acceleration sensor 37 and the gyrosensor 38, the attitude of the electronic device 1 affixed to the waist of the user. The attitude estimator 53 includes a gravitational direction estimator/corrector 53a, and a direction of travel attitude estimator 53b. The gravitational direction estimator/corrector 53a estimates an incline relative to the gravitational direction, and converts the estimated incline to data along an axis relative to the gravitational direction. Additionally, the direction of travel attitude estimator 53b estimates an incline relative to the direction of travel in which the user runs.

The left-right movement estimator 54 estimates movement in the left-right direction (the x-axis direction) of the electronic device 1 affixed to the waist of the user. That is, the left-right movement estimator 54 estimates left-right direction movement of the user. The left-right movement estimator 54 includes a direction of travel attitude corrector 54a, a velocity data corrector 54b, and a position data corrector 54c. The direction of travel attitude corrector 54a corrects the incline relative to the direction of travel in which the user runs, which is estimated by the direction of travel attitude estimator 53b, so that the y axis is aligned so as to be parallel to the direction of travel. The velocity data corrector 54b obtains an integration error included in the velocity data calculated by integrating the acceleration data, and subtracts the integration error from the velocity data to correct the velocity data. The position data corrector Mc obtains an integration error included in the position data calculated by integrating the velocity data corrected by the velocity data corrector 54b, and subtracts the integration error from the position data to correct the position data.

Next, a control method (exercise data acquisition method) of the electronic device 1 is described while referencing the drawings. Here, the series of exercise data acquisition methods described hereinafter is realized by the central control circuit 31 described above executing the predetermined control programs.

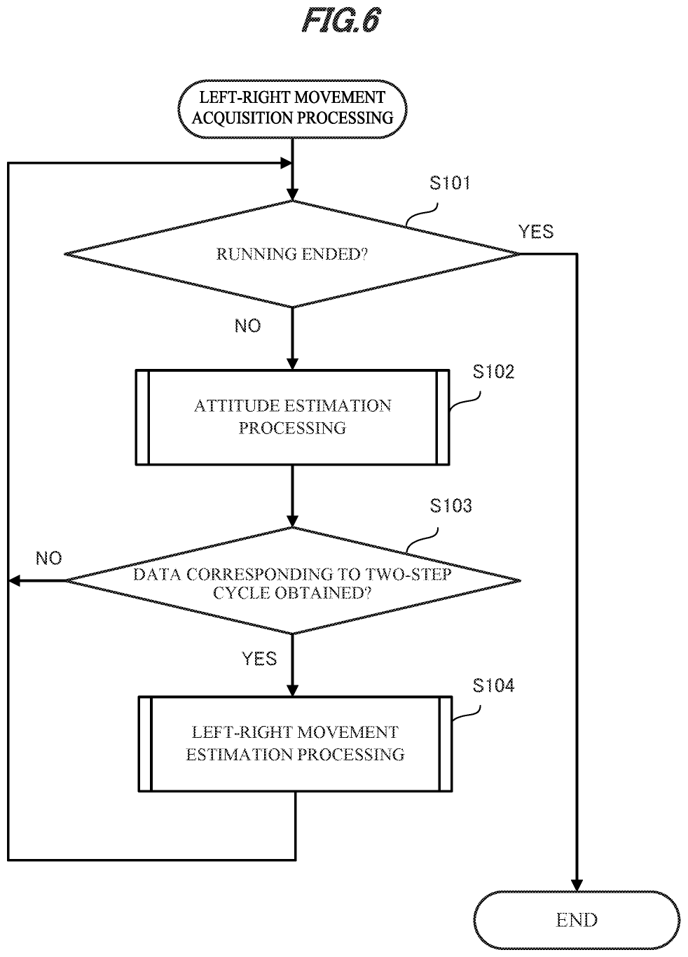

Firstly, an overview of a left-right movement data acquisition method of the electronic device 1 according to the present embodiment is described. FIG. 6 is a flowchart illustrating left-right movement acquisition processing of the electronic device 1 according to the present embodiment. The user affixes the electronic device 1 to the waist and operates the power key 2, thereby setting the electronic device 1 to a state capable of measuring exercise data and, then, starts running. When the running starts, sensor signals from the acceleration sensor 37 and the gyrosensor 38 are output. When the sensor signals are output, the acceleration data acquirer 51 samples the sensor signals of the acceleration sensor 37 on a predetermined cycle such as, for example, a sampling frequency of 200 Hz, and stores the acceleration data in the storage 34. Thus, the acceleration data is acquired. Likewise, the angular velocity data acquirer 52 samples the sensor signals of the gyrosensor 38 at the sampling frequency of 200 Hz, and stores the angular velocity data in the storage 34. Thus, the angular velocity data is acquired.

The controller 50 monitors the exercise state on the basis of the acceleration signal detected from the acceleration sensor 37 or the angular velocity signal detected from the gyrosensor 38, and determines whether the running is continuing or is ended (step S101). For example, when an acceleration signal greater than or equal to a predetermined value is detected in a predetermined interval, a determination is made that the running is continuing and, when not detected, a determination is made that the running is ended. When the controller 50 determines that the running is continuing (step S101; NO), the controller 50 performs attitude estimation processing every time the sensor data is obtained (step S102). Additionally, the controller 50 simultaneously determines, from the acceleration and the like of the sensor data, whether the legs have moved two steps as the running state of the user (step S103), and as a result of acceleration data corresponding to a two-step cycle (x-axis direction acceleration data) being acquired and stored in the memory, determines that the acceleration data corresponding to the two-step cycle is obtained (step S103; YES). When a determination is made that the x-axis direction acceleration data corresponding to the two-step cycle is obtained, the controller 50 performs left-right movement estimation processing for estimating the left-right direction movement of the user (step S104). After the left-right movement estimation processing is executed, step S101 is executed. When the x-axis direction acceleration data corresponding to the two-step cycle is not obtained (step S103; NO), step S101 is executed and, as long as the running is continuing, the operation of executing step S101 is repeated until the x-axis direction acceleration data corresponding to the two-step cycle is obtained. Additionally, in step S101, when a determination is made that the running is ended (step S101; YES), the left-right movement acquisition processing is ended.

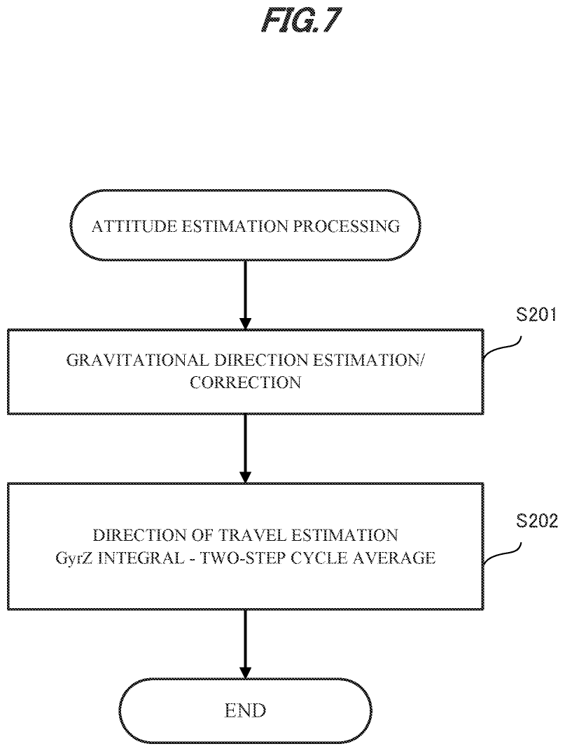

Next, the attitude estimation processing is described while referencing FIG. 7. As described above, the inertia sensors, including the acceleration sensor 37 and the gyrosensor 38, are affixed to the waist of the user. Here, when running is being performed, the attitude of the user may lean forward or lean to the left or right. In such a case, the z-axis direction that is intended to be parallel to the gravitational direction and the y-axis direction that is intended to be parallel to the direction of travel of the user are inclined amounts corresponding to angles of inclination of the waist of the user to which the inertia sensors are affixed. As such, attitude estimation processing is performed for estimating these inclines on the basis of the data of the acceleration sensor 37 and the gyrosensor 38.

In the attitude estimation processing, the controller 50 firstly estimates the incline relative to the gravitational direction, and performs gravitational direction estimation/correction processing for converting to data along the axis relative to the gravitational direction, that is, to axis coordinate data that has the y axis and the x axis, along the horizontal direction and the gravitational direction as the z-axis direction (step S201), One example of an estimation method includes inputting the three-axis output of the acceleration sensor 37 and the three-axis output of the gyrosensor 38 into a Kalman filter or a low pass filter to calculate three-axis data of the acceleration and three-axis data of the angular velocity relative to the ground, and estimate the gravitational direction. A configuration is possible in which an axis estimation method other than the Kalman filter or low-pass filter is used to perform estimation of the gravitational direction. When the estimation of the gravitational direction is performed, the attitude is corrected to the gravitational direction estimated for the data of the acceleration sensor 37 and the gyrosensor 38. As a result of this processing, the z-axis direction of the data of the acceleration sensor 37 and the gyrosensor 38 faces the gravitational direction.

Next, the controller 50 performs direction of travel estimation processing for estimating the incline relative to the direction of travel in which the user runs, and aligning the y axis so as to be parallel to the direction of travel (step S202). Angle data is obtained by integrating the angular velocity data from the gyrosensor 38 that is subjected to the attitude correction processing of the gravitational direction in the direction of travel estimation processing, and a difference between the current y-axis direction of the gyrosensor 38 and the direction of travel is calculated to perform estimation of the direction of travel and perform direction of travel attitude correction. However, because the angle is obtained by integrating the angular velocity data, integration error occurs, and error also occurs when the user turns at a curve or the like while running. It is thought that the results shift due to the accumulation of these errors.

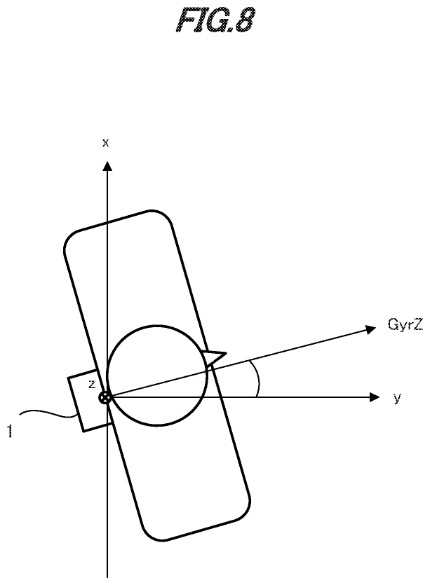

This state is described while referencing FIG. 8. FIG. 8 illustrates a state in which the user is running, and illustrates a state in which the user is shifted an angular velocity data GyrZ in the z-axis direction with respect to the direction of travel. While running, the user extends the legs in an alternating manner and twists at the waist. As such, the waist alternately rotates left and right, and the angular velocity data GyrZ about the z axis draws a sine wave centered on the direction of travel. However, when, for example, the user is running and turning at a curve, a shift occurs in the center of the angular velocity.

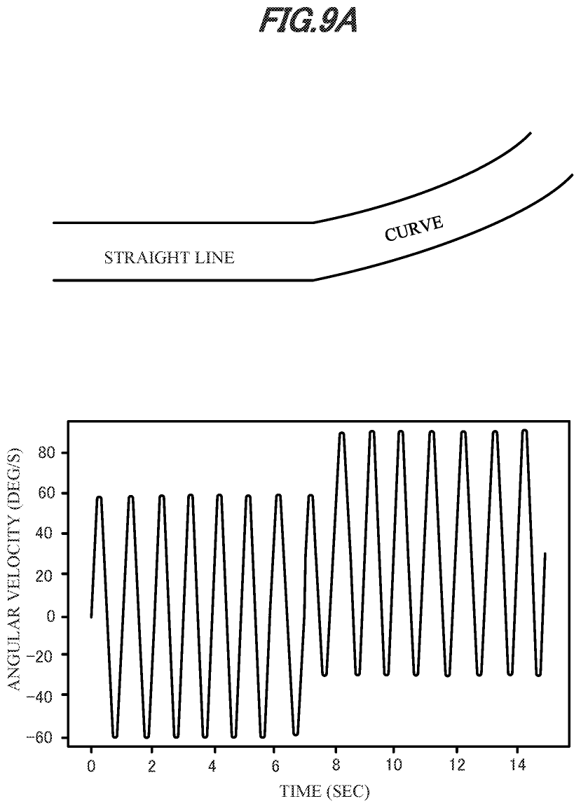

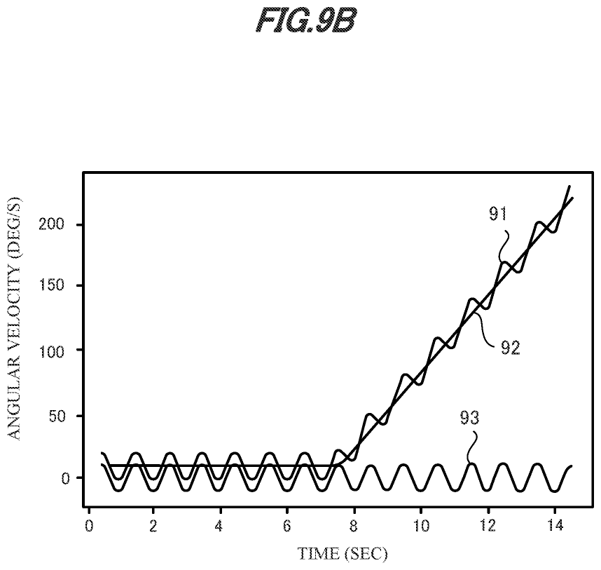

FIG. 9A illustrates the angular velocity data GyrZ about the z axis in a state in which the user is running along a curve from a straight line. Here, in the state in which the user is running along the curve, a shift occurs in the center of the angular velocity. The curve 91 of FIG. 9B illustrates a case in which this angular velocity data is integrated and illustrated as angle data. Originally, the angular velocity is as illustrated by the curve 93, but a large shift occurs in the angle due to the user turning at the curve.

Thus, the direction of travel changes moment by moment and direction of travel attitude error increases in accordance with this change. When the direction of travel attitude error increases, the y-axis is not corrected to the correct direction and, as a result, the x-axis also is not corrected to the correct direction. That is, precisely calculating the left-right direction movement becomes impossible. Therefore, this attitude error is extracted and removed from the angle data to correct the y axis to the correct direction. In the present embodiment, this correction is performed using the angular velocity data corresponding to the two-step cycle when walking or running.

Generally, in a running motion, a total of two steps including one step each of the left and right feet can be defined as one cycle. In this cycle, one foot, for example the right foot, steps forward in the direction of travel and strikes, and then the one foot kicks off (ground departure of the right foot) and, then, the other foot (left foot) strikes, the other foot kicks off (ground departure of the left foot), and the one foot (the right foot) strikes again.

At this time, the waist moves right-wise from the direction of travel with respect to the kicking of the right foot. Thereafter, the waist reverses and move left-wise due to the striking of the right foot and returns to the direction of travel by the time the right foot kicks and, furthermore, moves left-wise, reverses and moves right-wise due to the striking of the left foot, and returns to the direction of travel.

That is, the locus of angle around the z axis in this period is left-right symmetrical, and, when averaged, is 0. Accordingly, when the error is absent, the average of the angles of the two-step cycle is 0 and, conversely, when the error is present, the average of the angles of the two-step cycle indicates the direction of travel attitude error.

As such, an average of the integration results of the two-step cycle is calculated and subtracted from the integration results. As a result, velocity data from which the attitude error is removed is obtained. Thus, the difference between the direction of travel and the y-axis direction of the sensors can be made an average of 0 over the two-step cycle.

The two-step cycle is obtained on the basis of the angular velocity data from the gyrosensor 38. When the user runs, the angular velocity is 0 when the left foot is extended in the direction of travel and the heel strikes the ground surface. The angular velocity increases in the − direction as a result of the left foot kicking off backward after the heel of the left foot strikes the ground surface. The angular velocity is at a maximum when the user is facing the direction of travel. The angular velocity decreases as the right foot moves in the direction of travel after the user is facing the direction of travel. When the heel of the right foot strikes, the angular velocity is 0.

The angular velocity increases in the + direction as a result of the right foot kicking off backward after the heel of the right foot strikes. The angular velocity is at a maximum when the user is facing the direction of travel. The angular velocity decreases as the left foot moves in the direction of travel after the user is facing the direction of travel. When the heel of the left foot strikes, the angular velocity is 0. Thus, the two-step cycle can be obtained from the timing at which the angular velocity is 0 or from the timing at which the angular velocity is at a maximum or a minimum.

Alternatively, the two-step cycle can be obtained from the acceleration data from the acceleration sensor 37. In a series of running motions, a vertical direction acceleration component of the acceleration data acquired by the acceleration sensor 37 demonstrates a signal waveform having periodicity for every left-right step. From this signal waveform, the two-step cycle in the vertical direction acceleration component corresponds to one cycle of the running motion. Accordingly, it is possible to stably cut out, on the basis of the vertical direction acceleration component acquired by the acceleration sensor, motion data for every two-step cycle in the running motions performed by the user. In addition, it is possible to accurately measure the time of the two-step cycle. For example, the + direction acceleration is at a maximum at the timing at which the heel of the right foot strikes, and the − direction acceleration is at a maximum at the timing at which the heel of the left foot strikes. Note that another method may be used to as the cycle estimation method.

Before performing the direction of travel estimation processing, angular velocity data corresponding to the two-step cycle must be obtained. The angular velocity signal detected from the gyrosensor 38 is input into the angular velocity data acquirer 52, and the angular velocity data acquirer 52 samples at the predetermined sampling cycle and stores the results in the storage 34. When angular velocity data corresponding to the two-step cycle is stored in the storage 34, the z-axis direction angular velocity data GyrZ corresponding to the two-step cycle is read from the storage 34. The z-axis direction angular velocity data GyrZ in this two-step cycle period is integrated, and angles are calculated. When the angles are calculated, an average of the angles in the two-step cycle period is calculated. The left and right swinging in the two-step cycle form a pair and, as such, the average of the angles is expected to be 0.

However, integration error and shifting caused by curves and the like appear in this average. Curve 92 of FIG. 9B is a curve that expresses the average. Here, the average is obtained by performing linear interpolation between the average value of the two-step cycle and an average value of the two-step cycle centered one step before. Note that, the average values used in the linear interpolation may be the average value of the two-step cycle, and an average value of an adjacent two-step cycle centered two steps before. Moreover, the present disclosure is not limited to the two-step cycle, and an integer multiple of the two-step cycle such as a four-step cycle, a six-step cycle, or the like, may be used. The controller 50 obtains the averages described above to calculate the shift caused by the integration error and the like, subtracts this shift from the angle obtained by integrating the velocity data, and corrects the direction of travel. Thus, the attitude estimation processing is ended.

In FIG. 7, when the attitude estimation processing is performed and the acceleration data corresponding to the two-step cycle is stored in the storage 34, the acceleration data corresponding to the two-step cycle is read from the storage 34 and left-right movement estimation processing is performed (FIG. 6, step S104).

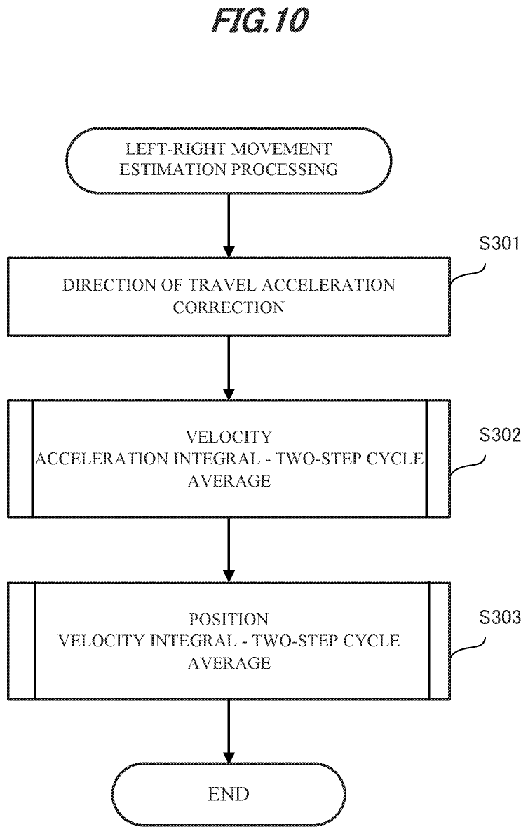

Next, the left-right movement estimation processing is described while referencing FIG. 10.

Firstly, the direction of travel attitude correction, using the direction of travel attitude error obtained in the attitude estimation processing, is performed on the stored acceleration data corresponding to the two-step cycle. As a result, the direction of travel attitude correction is performed in addition to the gravitational direction attitude correction already performed by the attitude estimation processing, and acceleration for which the z axis is along the gravitational direction and the y axis is along the direction of travel, that is, acceleration for which the x-axis direction is also attitude-corrected, is acquired (step S301).

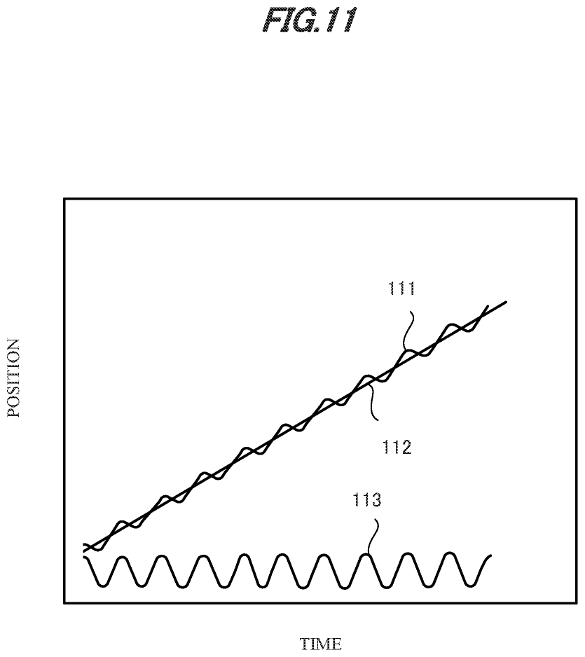

The acceleration data on which the direction of travel attitude correction is performed in step S301 is subjected to integration processing, and velocity is calculated. However, integration error occurs as a result of performing the integration processing, and the calculated velocity data includes this integration error. In order to calculate the position data, the velocity data must be integrated again. Further integration error occurs as a result of integrating the velocity data, and the error of the calculated position data increases. FIG. 11 illustrates position data for a case in which the acceleration data is integrated two times, and curve 111 expresses the position data that is integrated two times. The error increases due to the accumulation of the integration error caused by the integration of the acceleration data and the integration error caused by the integration of the velocity data. As such, as in the case of the direction of travel attitude correction described above, this error component is calculated by obtaining the average of the integration data corresponding to the two-step cycle. As described above, the locus of angle around the z axis in the two-step cycle is left-right symmetrical, and, when averaged, is 0. Likewise, changes in the left-right direction velocity data, that is, the x-axis direction velocity data, and changes in the left-right direction position data, that is, the x-axis direction position data are left-right symmetrical and, when averaged, are 0. Accordingly, when the integration error is absent, the averages of the x-axis direction velocity data and position data of the two-step cycle are 0 and, conversely, when the error exists, the averages of the x-axis direction velocity data and position data of the of the two-step cycle express integration error.

Here, the error component is calculated by obtaining the average for every two-step cycle, and performing linear interpolation between the obtained average and a subsequently obtained average for the velocity data and the position data. In step S302, the acceleration data that is subjected to the direction of travel attitude correction is subjected to integration processing to calculate the velocity data and obtain the error component of the velocity data, and the error component is subtracted from the velocity data to obtain velocity data for which the error is corrected.

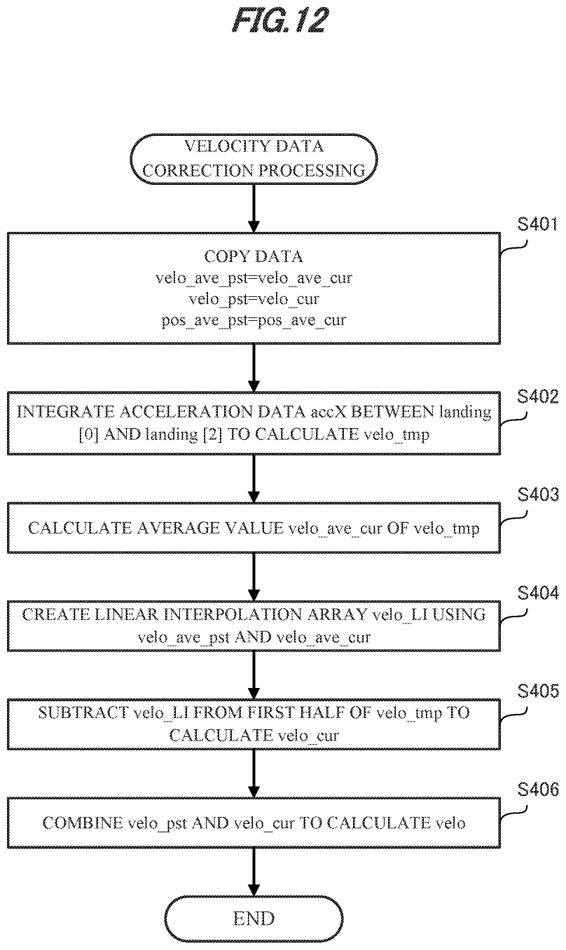

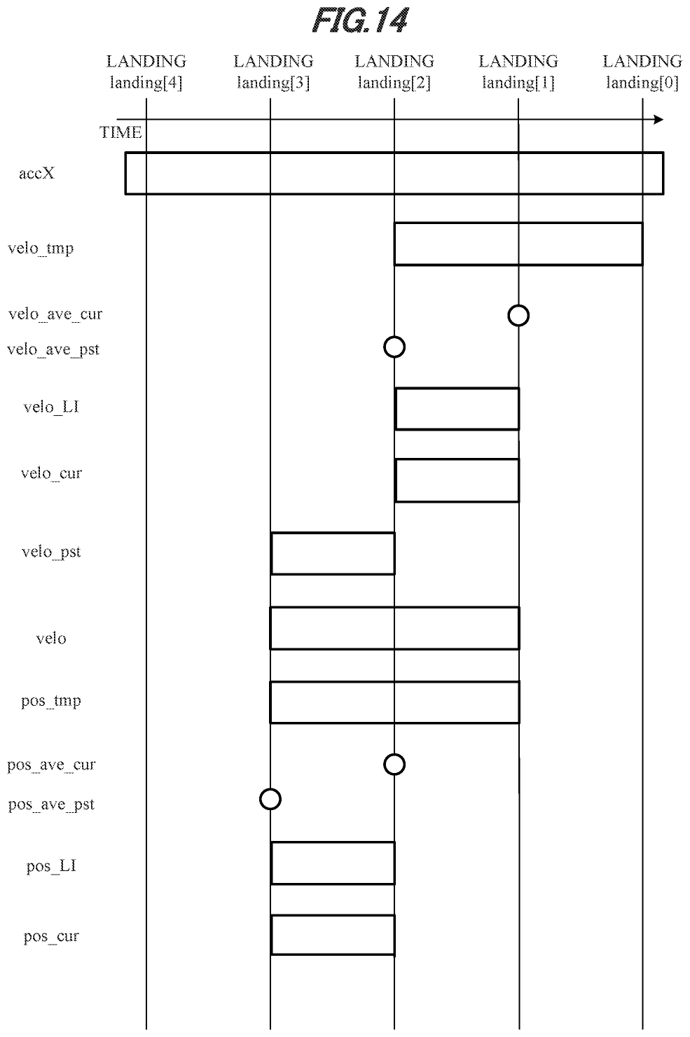

Next, velocity data correction processing in step S302 is described while referencing FIGS. 12 and 14. In the time chart of FIG. 14, the horizontal axis, namely the time axis, illustrates, into the past from right to left, a landing [0] that is a most recent landing, a landing [1] that is a landing one step before, a landing [2] that is a landing two steps before, a landing [3] that is a landing three steps before, and a landing [4] that is a landing four steps before.

The correction processing is performed every one step landing, and acceleration data accX, velocity data velo_tmp, an average value velo_ave_cur of the velocity data of a period of the two-step cycle obtained most recently, an average value velo_ave_pst of the velocity data of a period of the two-step cycle obtained last time, an interpolated value velo_LI of the velocity data interpolated on the basis of both of the average values, corrected velocity data velo_cur corrected on the basis of both of the average values most recently obtained, a corrected velocity data velo_pst obtained last time, position data pos_tmp, an average value pos_ave_cur of the position data of the period of the two-step cycle obtained most recently, an average value pos_ave_pst of the position data of the period of the two-step cycle obtained last time, an interpolated value pos_LI of the position data interpolated on the basis of both of the average values, corrected position data pos_cur corrected on the basis of both of the average values, and the like are stored in the storage 34 as parameters.

Currently, data based on the acceleration data up to the landing [1] that is before the landing of landing [0] is stored as the aforementioned parameters in the storage 34. Specifically, the average value of the velocity data in the range between the landing [1] and the landing [3] that is the two-step cycle before the landing [1] is stored as the average value velo_ave_cur of the period of the two-step cycle obtained most recently, the corrected velocity data between the landing [2] and the landing [3] is stored as the corrected velocity data velo_cur obtained most recently, and the average value of the position data in the range between the landing [2] and the landing [4] that is the two-step cycle before the landing [2] is stored as the average value pos_ave_cur of the position data of the period of the two-step cycle obtained most recently.

Here, when the acceleration data of the landing [0] is newly input, updating is performed for the velocity data correction processing.

In FIG. 12, the controller 50 reads out, from the storage 34, the average value velo_ave_cur of the velocity data of the period of the two-step cycle obtained most recently, the corrected velocity data velo_cur obtained most recently, and the average value pos_ave_cur of the position data of the period of the two-step cycle obtained most recently. The controller 50 data copies the average velocity data between the landing [1] and the landing [3], read out as the average value velo_ave_cur of the velocity data of the period of the two-step cycle obtained most recently, to the average value velo_ave_pst of the velocity data of the period of the two-step cycle obtained last time. Additionally, the controller 50 data copies the corrected velocity data between the landing [2] and the landing [3], read out as the corrected velocity data velo_cur obtained most recently, to the corrected velocity data velo_pst obtained last time. Furthermore, the controller 50 data copies the average position data between the landing [2] and the landing [4], read out as the average value pos_ave_cur of the position data of the period of the two-step cycle obtained most recently, to the average value pos_ave_pst of the position data of the period of the two-step cycle obtained last time (step S401).

Next, the controller 50 integrates the acceleration data accX in the range between the landing [0] and the landing [2] to obtain the velocity data velo_tmp at each point in time (step S402). In one example, this velo_tmp is obtained as an array of velocity data for every 5 ms.

When the velocity data velo_tmp between the landing [0] and the landing [2] is obtained, the controller 50 obtains, as the average value velo_ave_cur of the velocity data of the period of the two-step cycle obtained most recently, the average value of the velocity data in the range of two-step cycle of the landing [0] and the landing [2], and stores the result in the storage 34 (step S403). Here, the period of the two-step cycle obtained most recently is an example of a period that is a multiple of the first two-step cycle or a period that is a multiple of the second two-step cycle. As described above, the average of the velocity data of the period of the two-step cycle is expected to be 0, but error occurs due to integration error and the like. As such, the velocity data is corrected by subtracting the average value of the velocity data of the period of the two-step cycle from the velocity data.

When the velo_ave_cur is obtained, the controller 50 calculates the interpolated value using the velo_ave_cur and the average value velo_ave_pst of the velocity data of the period of the two-step cycle obtained last time that is the average value of the velocity data of the period of the two-step cycle of the landing [1] and the landing [3] data copied in step S401 (step S404). Here, the period of the two-step cycle obtained last time is an example of a period that is a multiple of the second two-step cycle or a period that is a multiple of the first two-step cycle. The velo_ave_pst, which is the average value of the velocity data of the period of the two-step cycle of landing [1] and landing [3], is the average value of the period of the two-step cycle centered on the landing [2] that is one step before the velo_ave_cur, and is set such that the periods of the two-step cycle of the velo_ave_pst and the velo_ave_cur partially overlap each other between the landing [1] and the landing [2] on the time axis. Accordingly, the average value of the velocity data of the period of the two-step cycle at each point in time between the landing [1] and the landing [2] is interpolated using the values of the average value velo_ave_cur of the velocity data of the period of the two-step cycle centered on the point in time of the landing [1] and the average value velo_ave_pst of the velocity data of the period of the two-step cycle centered on the point in time of the landing [2]. In this case, linear interpolation is used as the interpolation. The average value velo_ave_cur of the velocity data at the point in time of the landing [1] and the average value velo_ave_pst of the velocity data at the point in time of the landing [2] are connected by a straight line, and the average value of the velocity data at each point in time between the average value velo_ave_cur and the average value velo_ave_pst is obtained. As with the velo_tmp, in one example, the average value velo_LI of the linearly interpolated velocity data at each point in time is obtained as an array of average values of the velocity data for every 5 ms.

When the array velo_LI of the average values of the linearly interpolated velocity data at each point in time between the landing [1] and the landing [2] is obtained, the controller 50 removes the average value of the velocity data from the velocity data velo_tmp obtained in step S402 to obtain the corrected velocity data velo_cur that is corrected (step S405). Of the velocity data velo_tmp obtained in step S402, the velocity data at each point in time between the landing [1] and the landing [2] is used for the velo_tmp. From the velocity data velo_tmp at each point in time between the landing [1] and the landing [2], the respectively corresponding average value velo_LI of the linearly interpolated velocity data at each point in time is subtracted and, as a result, the corrected velocity data velo_cur that is corrected at each point in time between the landing [1] and the landing [2] is obtained.

The controller 50 combines the obtained velocity data velo_cur corrected at each point in time between the landing [1] and the landing [2] with the corrected velocity data velo_pst between the landing [2] and the landing [3] that is data copied in step S401 to obtain the corrected velocity data velo corresponding to the two-step cycle of the landing [1] and the landing [3] (step S406). Thus, the velocity data correction processing is ended.

Returning to FIG. 10, the corrected velocity data subjected to the integration error correction in step S302 is subjected to integration processing and, as a result, the position data is calculated. As described above, integration error occurs as a result of the integration processing being performed on the velocity data, and the calculated position data includes this integration error. The average value of the position data of the period of the two-step cycle is expected to be 0, but error occurs due to the integration error and the like. As such, corrected position data is obtained by removing, from the position data obtained by integrating the corrected velocity data, the average value of the position data of the period of the two-step cycle centered on that point in time (step S303).

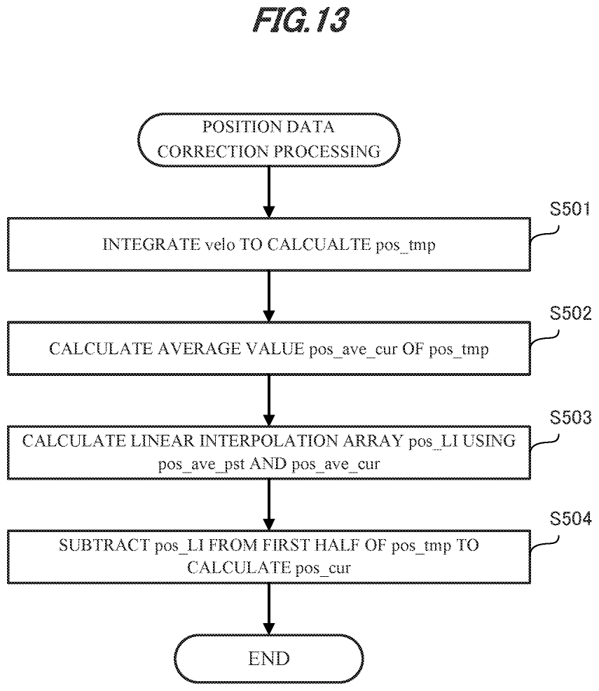

Next, the position data correction processing of step S303 is described while referencing FIG. 13 and the aforementioned time chart of FIG. 14.

The controller 50 obtains the position data pos_tmp by integrating the corrected velocity data velo, corrected in the velocity data correction processing of FIG. 12 described above, corresponding to the two-step cycle of the landing [1] and the landing [3] (step S501). In one example, the pos_tmp is obtained as an array of the position data for every 5 ms.

When the position data pos_tmp between the landing [1] and the landing [3] is obtained, the controller 50 obtains, as the average value pos_ave_cur of the position data of the period of the two-step cycle obtained most recently, the average value of the position data of the period of the two-step cycle of the landing [1] and the landing [3] (step S502). As described above, the average of the two-step cycle is expected to be 0, but error occurs due to integration error and the like. As such, the position data is corrected by subtracting the average value of the position data of the period of the two-step cycle from the position data.

When the pos_ave_cur is obtained, the controller 50 calculates the interpolated value using this pos_ave_cur and the average value of the position data of the period of the two-step cycle of the landing [2] and the landing [4] data copied to the average value pos_ave_pst of the position data of the period of the two-step cycle obtained last time in step S401 of FIG. 11 (step S503). The pos_ave_pst, which is the average value of the position data of the period of the two-step cycle of the landing [2] and the landing [4], is the average value of the position data of the period of the two-step cycle centered on the landing [3] that is one step before the pos_ave_cur, and is set such that the periods of the two-step cycle of the pos_ave_pst and the pos_ave_cur partially overlap each other between the landing [2] and the landing [3] on the time axis. Accordingly, the average value of the position data of the period of the two-step cycle at each point in time between the landing [2] and the landing [3] is interpolated using the values of the average value pos_ave_cur of the position data of the period of the two-step cycle centered on the point in time of the landing [2] and the average value pos_ave_pst of the position data of the period of the two-step cycle centered on the point in time of the landing [3]. In this case, as with the velocity, linear interpolation is used as the interpolation. The average value pos_ave_cur of the position data of the period of the two-step cycle centered at the point in time of the landing [2] and the average value pos_ave_pst of the position data of the period of the two-step cycle centered at the point in time of the landing [3] are connected by a straight line, and the average value of the position data of the period of the two-step cycle at each point in time between the average value pos_ave_cur and the average value pos_ave_pst is obtained. As with the pos_tmp, in one example, the average value pos_LI of the linearly interpolated position data of the period of the two-step cycle at each point in time is obtained as an array of average values of the two-step cycle for every 5 ms.

When the average value pos_LI of the linearly interpolated position data of the period of the two-step cycle at each point in time between the landing [2] and the landing [3] is obtained, the controller 50 removes the average value of the position data of the period of the two-step cycle from the position data pos_tmp obtained in step S501 to obtain the corrected position data pos_cur that is corrected (step S504). Of the position data pos_tmp obtained in step S501, the position data at each point in time between the landing [2] and the landing [3] is used for the pos_tmp. From the position data pos_tmp at each point in time between the landing [2] and the landing [3], the respectively corresponding average value pos_LI of the linearly interpolated position data of the two-step cycle at each point in time is subtracted and, as a result, the corrected position data pos_cur at each point in time between the landing [2] and the landing [3] is obtained. Thus, the position data correction processing is ended.

As described above, when obtaining the velocity data by integrating the acceleration data, the average of the velocity data corresponding to the two-step cycle is subtracted to obtain the velocity data for which the error is corrected and, also, when obtaining the left-right movement position data by integrating the corrected velocity data, the average corresponding to the two-step cycle is subtracted to obtain the left-right movement position data for which the error is corrected. As a result, it is possible to reduce the error of the left-right movement position data caused by the accumulation of integration error resulting from integrating two times, and it is possible to precisely calculate the left-right movement position data.

Moreover, the velocity data and the position data are corrected by linear interpolation on the basis of the averages corresponding to the two-step cycle and, as such, the error can be stably and precisely corrected. The processing for correcting by linear interpolation is simple and, as such, memory capacity required for the processing is small, the amount of calculation is small, and the load on the CPU can be reduced. Additionally, since the processing is simple, the processing can respond immediately, even when the two-step cycle changes rapidly such as in a case in which the user trips while running.

In the embodiment described above, the error component caused by the integration error and the like of the velocity data and the position data is obtained by linearly interpolating, on the basis of the average value of the two-step cycle and the average value of a two-step cycle centered one step before, between the average values. However, the present disclosure is not limited thereto, and a configuration is possible in which the average value of the two-step cycle and the average value of a continuous two-step cycle centered two steps before are used for the average values used in the linear interpolation and are set such that the two-step cycles partially overlap each other on the time axis, or are set such that the two-step cycles do not partially overlap each other and are continuous with each other without a spacing. Additionally, the average values used in the linear interpolation are the average values of the two-step cycle, but the present disclosure is not limited thereto, and a configuration is possible in which the average value of the velocity data or the average value of the position data of a period that is a multiple of the two-step cycle such as, for example, a four-step period, a six-step period, or the like is used. Furthermore, in such a case, portions of adjacent cycles used in the linear interpolation may overlap, or may be continuous without overlapping. For example, in the case of a four-step cycle, the adjacent four-step cycle may be a four-step cycles centered two steps before, or may be a four-step cycle centered four steps before. Additionally, the interpolation is not limited to linear interpolation based on two average values and, for example, may be quadratic interpolation based on three average values. Specifically, quadratic interpolation may be performed using three average values, namely the average value of the velocity data or the average value of the position data of a period of a first two-step cycle, the average value of the velocity data or the average value of the position data of a period of a second two-step cycle centered one step before, and the average value of the velocity data or the average value of the position data of a period of a third two-step cycle centered two steps before, and an interpolated value between these average values may be obtained. Note that, in addition, the interpolation may be based on four or more average values.

By integrating the angular velocity data to calculate the angle data and, also, correcting the direction of travel attitude by correcting the direction of travel angle data using the angle data error calculated by the value linear interpolated on the basis of the average of the two-step cycle of the angle data, the attitude can be corrected in correspondence with the direction of travel that changes from moment to moment and, furthermore, the left-right movement position data can be stably and precisely calculated.

Note that, in the present embodiment, a description is given in which, after performing the attitude estimation processing and correcting the attitude, the left-right movement estimation processing is performed and the data of the left-right movement of the user is acquired. However, the present disclosure is not limited thereto, and a configuration is possible in which, for example, the attitude estimation processing is omitted and only the left-right movement estimation processing is performed. As described above, by performing the left-right movement estimation processing, the accumulation of integration error resulting from integrating the acceleration data two times can be eliminated, and the left-right movement position data can be calculated stably and precisely. Furthermore, by adding the attitude estimation processing as in the present embodiment, the attitude can be corrected in correspondence with the changes of the direction of travel and, furthermore, the left-right movement position data can be stably and precisely calculated.

The present embodiment describes a case in which exercise data is acquired for a case in which the user is walking or running. However, the present disclosure is not limited thereto, and the present embodiment may be applied to a case in which exercise data of cycling or the like is recorded.

In the present embodiment, the electronic device 1 includes the acceleration sensor 37 and the gyrosensor 38, but a configuration is possible in which the acceleration sensor 37 and the gyrosensor 38 are provided separately from the electronic device 1. In such a case, the acceleration sensor 37 and the gyrosensor 38 are affixed to the waist of the user, and are wiredly or wirelessly connected to the electronic device 1.

In the present embodiment, the electronic device 1 acquires the sensor data from the acceleration sensor 37 and the gyrosensor 38, corrects the left-right movement exercise data, and transmits the corrected left-right movement exercise data wirelessly to the external device 41, and the external device 41 performs the analysis of the data and displays the analysis results. Accordingly, the electronic device 1 and the external device 41 constitute a system for acquiring and analyzing exercise data. However, the present disclosure is not limited thereto, and a configuration is possible in which the electronic device 1 performs the analysis of the data and transmits the analysis results to the external device 41. Additionally, a configuration is possible in which the electronic device 1 includes a display such as a liquid crystal display or the like, and displays display results.

Conversely, a configuration is possible in which the electronic device 1 only acquires the sensor data from the acceleration sensor 37 and the gyrosensor 38, and the external device 41 performs the correction processing of the left-right movement exercise data.

A configuration is possible in which the electronic device 1 includes a card slot, for example, a non-transitory recording medium such as a memory card is detachably provided, and the acquired data and corrected data are stored on this non-transitory recording medium.

The electronic device 1 includes the acceleration sensor 37 and the gyrosensor 38 as sensors, but a configuration is possible in which the electronic device 1 further includes a geomagnetic sensor, a GPS receiver, or the like.

In the embodiment described above, the central control circuit 31 executes the programs stored in the ROM 32 to function as the controller 50. However, a configuration is possible in which, instead of the central control circuit 31 executing the programs stored in the ROM 32, an Application Specific Integrated Circuit (ASIC), a Field-Programmable Gate Array (FPGA), various control circuitry, or other dedicated hardware is provided, and this dedicated hardware functions as the controller In such a case, the controller may be realized in part by dedicated hardware and in part by software or firmware.

In the embodiment described above, the programs are stored in advance in the ROM 32, but a configuration is possible in which the programs are read out, via a non-transitory recording medium acquirer, to the RAM 33 or the like from an external non-transitory recording medium such as a memory card or the like, and stored. In addition, the programs can be superimposed on a carrier wave and be read out to the RAM 33 or the like via a communication medium such as the internet, and stored.

The foregoing describes some example embodiments for explanatory purposes. Although the foregoing discussion has presented specific embodiments, persons skilled in the art will recognize that changes may be made in form and detail without departing from the broader spirit and scope of the invention.

This application claims the benefit of Japanese Patent Application No. 2020-153469, filed on Sep. 14, 2020, the entire disclosure of which is incorporated by reference herein.

### INDUSTRIAL APPLICABILITY

The present disclosure is applicable to an electronic device, an exercise data acquisition method, and a program that generates exercise data using inertia sensors.

### REFERENCE SIGNS LIST

1 Electronic device
2 Power key
3 Display
4 Clip
31 Central control circuit
32 ROM
33 RAM
34 Storage
35 Wireless communication module
36 Input/output control circuit
37 Acceleration sensor
38 Gyrosensor
39 Time keeper
40 Power supply circuit
41 External device
50 Controller
51 Acceleration data acquirer
52 Angular velocity data acquirer
53 Attitude estimator
53a Gravitational direction estimator/corrector
53b Direction of travel attitude estimator
54 Left-right movement estimator 54
54a Direction of travel attitude corrector
54b Velocity data corrector
54c Position data corrector
91, 92, 93, 111, 112, 113 Curve

1. An electronic device, comprising:
a controller,
wherein the controller is configured to:
acquire, from an acceleration sensor, acceleration data corresponding to a motion state in a body-lateral direction orthogonal to a body axis of a user that is moving by legs of the user, and
derive velocity data based on the acceleration data and, also, derive an error of the velocity data based on an average value of the velocity data of a period that is a multiple of a first two-step cycle and an average value of the velocity data of a period that is a multiple of a second two-step cycle that directly precedes or follows the period that is a multiple of the first two-step cycle, and correct the velocity data using the error of the velocity data to generate corrected velocity data.

2. The electronic device according to claim 1, wherein the controller is configured to derive position data based on the corrected velocity data and, also, derive an error of the position data based on an average value of the position data of a period that is a multiple of the first two-step cycle and an average value of the position data of a period that is a multiple of the second two-step cycle that directly precedes or follows the period that is a multiple of the first two-step cycle, and correct the position data using the error of the position data to generate corrected position data.

3. An electronic device comprising:
a controller,
wherein the controller is configured to:
acquire, from an acceleration sensor, acceleration data corresponding to a motion state in a body-lateral direction orthogonal to a body axis of a user that is moving by legs of the user, and
derive position data based on the acceleration data and, also, derive an error of the position data based on an average value of the position data of a period that is a multiple of a first two-step cycle and an average value of the position data of a period that is a multiple of a second two-step cycle that directly precedes or follows the period that is a multiple of the first two-step cycle, and correct the position data using the error of the position data to generate corrected position data.

4. The electronic device according to claim 1, wherein the controller is configured to derive the error of the velocity data by linearly interpolating the average value of the velocity data of the period that is a multiple of the first two-step cycle and the average value of the velocity data of the period that is a multiple of the second two-step cycle.

5. The electronic device according to claim 1, wherein the controller is configured to:
obtain the average value of the velocity data of a period that is a multiple of a third two-step cycle continuous with the period that is a multiple of the second two-step cycle, and
derive the error of the velocity data by quadratically interpolating the average value of the velocity data of the period that is a multiple of the first two-step cycle, the average value of the velocity data of the period that is a multiple of the second two-step cycle, and the average value of the velocity data of the period that is a multiple of the third two-step cycle.

6. The electronic device according to claim 3, wherein the controller is configured to derive the error of the position data by linearly interpolating the average value of the position data of the period that is a multiple of the first two-step cycle and the average value of the position data of the period that is a multiple of the second two-step cycle.

7. The electronic device according to claim 3, wherein the controller is configured to:
obtain the average value of the position data of a period that is a multiple of a third two-step cycle continuous with the period that is a multiple of the second two-step cycle, and
derive the error of the position data by quadratically interpolating the average value of the position data of the period that is a multiple of the first two-step cycle, the average value of the position data of the period that is a multiple of the second two-step cycle, and the average value of the position data of the period that is a multiple of the third two-step cycle.

8. The electronic device according to claim 1, wherein the period that is a multiple of the first two-step cycle and the period that is a multiple of the second two-step cycle are set so as to partially overlap each other on a time axis.

9. The electronic device according to claim 1, wherein the period that is a multiple of the first two-step cycle and the period that is a multiple of the second two-step cycle are set so as not to overlap each other on a time axis and, also, to be continuous with each other without a spacing.

10. The electronic device according to claim 1, further comprising:
an attacher that is affixed to a waist of the user.

11. The electronic device according to claim 1, further comprising:
the acceleration sensor.

12. An exercise data acquisition method, comprising:
acquiring, from an acceleration sensor, acceleration data corresponding to a motion state in a body-lateral direction orthogonal to a body axis of a user that is moving by legs of the user;
deriving velocity data based on the acceleration data;
deriving an error of the velocity data based on an average value of the velocity data of a period that is a multiple of a first two-step cycle and an average value of the velocity data of a period that is a multiple of a second two-step cycle that directly precedes or follows the period that is a multiple of the first two-step cycle; and
correcting the velocity data using the error of the velocity data to generate corrected velocity data.

13. An exercise data acquisition method, comprising:
acquiring, from an acceleration sensor, acceleration data corresponding to a motion state in a body-lateral direction orthogonal to a body axis of a user that is moving by legs of the user;
deriving position data based on the acceleration data;
deriving an error of the position data based on an average value of the position data of a period that is a multiple of a first two-step cycle and an average value of the position data of a period that is a multiple of a second two-step cycle that directly precedes or follows the period that is a multiple of the first two-step cycle; and
correcting the position data using the error of the position data to generate corrected position data.

14. A non-transitory computer-readable recording medium storing a program for causing a computer to execute operations comprising:
acquiring, from an acceleration sensor, acceleration data corresponding to a motion state in a body-lateral direction orthogonal to a body axis of a user that is moving by legs of the user;
deriving velocity data based on the acceleration data;
deriving an error of the velocity data based on an average value of the velocity data of a period that is a multiple of a first two-step cycle and an average value of the velocity data of a period that is a multiple of a second two-step cycle that directly precedes or follows the period that is a multiple of the first two-step cycle; and
correcting the velocity data using the error of the velocity data to generate corrected velocity data.

15. A non-transitory computer-readable recording medium storing a program for causing a computer to execute operations comprising:
acquiring, from an acceleration sensor, acceleration data corresponding to a motion state in a body-lateral direction orthogonal to a body axis of a user that is moving by legs of the user;
deriving position data based on the acceleration data;
deriving an error of the position data based on an average value of the position data of a period that is a multiple of a first two-step cycle and an average value of the position data of a period that is a multiple of a second two-step cycle that directly precedes or follows the period that is a multiple of the first two-step cycle; and
correcting the position data using the error of the position data to generate corrected position data.
